(19) United States
(12) Patent Application Publication
(10) Pub. No.: US 2022/0288271 A1
(43) Pub. Date: Sep. 15, 2022

(54) BIOADHESIVE COMPOSITION COMPRISING MUSSEL ADHESIVE PROTEIN AND PREPARATION METHOD THEREOF

(71) Applicant: Postech Research and Business Development Foundation, Pohang-si (KR)

(72) Inventors: Hyung Joon CHA, Pohang-si (KR); Jang Woo Yang, Pohang-si (KR); Kye IL Joo, Daegu (KR)

(21) Appl. No.: 17/692,494

(22) Filed: Mar. 11, 2022

(30) Foreign Application Priority Data
Mar. 12, 2021 (KR) 10-2021-0032773
Jan. 10, 2022 (KR) 10-2022-0003448

(51) Int. Cl. A61L 15/58; A61L 15/22; A61L 15/64; A61L 26/00; C07K 14/435

(52) U.S. Cl. CPC A61L 15/585; A61L 15/225; A61L 15/64; A61L 26/0014; A61L 26/0047; A61L 26/0052; A61L 26/008; A61L 26/009; C07K 14/43504

(57) ABSTRACT

The present disclosure relates to a bioadhesive composition including mussel adhesive protein; and at least one of polyacrylic acid and polymethacrylic acid; in which the mussel adhesive protein is linked to the at least one of polyacrylic acid and polymethacrylic acid by an amide bond and has a three-dimensional network structure and a method for preparing the same.

### CROSS-REFERENCE TO RELATED APPLICATIONS

This application is based on and claims priority from Korean Patent Application No. 10-2021-0032773, filed on Mar. 12, 2021, and Korean Patent Application No. 10-2022-0003448, filed on Jan. 10, 2022 with the Korean Intellectual Property Office, the disclosure of which is incorporated herein in its entirety by reference.

### TECHNICAL FIELD

The present disclosure relates to a bioadhesive composition containing a mussel adhesive protein and a method for preparing the same, and more particularly, to a bioadhesive composition containing a mussel adhesive protein and a method for preparing the same, for biomedical applications.

### BACKGROUND

Bioadhesive refers to a substance that has adhesion properties to various biological samples such as cell membranes, cell walls, lipids, proteins, DNA, growth factors, cells, tissues, etc. and is applicable to various biomedical fields such as tissue adhesives, hemostats, scaffolds for tissue engineering, carriers for drug delivery, tissue augmentation, wound healing agents, anti-adhesive agents, etc.

Such bioadhesive is required to have strong adhesion and crosslinking abilities and must maintain its function in a living body for a long period of time. Bioadhesive which is currently commercialized or put into practical use includes cyanoacrylate instant adhesives, fibrin glue, gelatin glue, polyurethane-based adhesives, etc.

However, there are significant limitations that bioadhesives using synthetic polymers exhibit very weak adhesion ability under an aqueous environment of the living body, and the cyanoacrylate-based bioadhesives induce side effects such as immune responses in the human body, etc.

Further, although fibrin-based bioadhesives practically applied to patients induce fewer adverse effects such as immune responses in the human body, they have very low adhesion ability, and their prices are also high, which is a limitation in applications. In the case of gelatin bioadhesives, formalin or glutaraldehyde used as a crosslinking agent also causes a crosslinking reaction with proteins in the living body, leading to tissue toxicity. Polyurethane-based bioadhesives have a problem that aromatic diisocyanate used as a synthetic raw material exhibits biotoxicity.

Meanwhile, the mussel, a marine life, may produce and secret adhesive proteins to firmly attach itself to a wet solid surface such as a rock in the sea so that the mussel is not affected by the impact of waves or the buoyancy effect of seawater. Mussel adhesive protein is known as a strong natural adhesive, and when compared to chemically synthesized adhesives, it exhibits about twice the tensile strength of most epoxy resins and has the flexibility to bend.

Further, the mussel adhesive protein has the ability to adhere to various surfaces such as plastic, glass, metal, Teflon, and biomaterials. It may even adhere to wet surfaces within minutes, which remains an unfinished task in the development of chemical adhesives.

Furthermore, the mussel adhesive protein is known to not attack human cells or cause an immune response, so it has great potential for application in medical fields such as adhesion of living tissue during surgery or adhesion of broken teeth. The mussel adhesive protein may also be used for cell surface adhesion. The cell surface adhesion technology is one of the very important technologies required for cell culture and tissue engineering and is very critical for promoting cell proliferation and differentiation.

Accordingly, various biomedical applications using mussel adhesive protein are required.

### SUMMARY

The present disclosure has been made in an effort to provide a bioadhesive composition including a mussel adhesive protein that may be used as a bioadhesive tape or film and may control the decomposition time.

Further, the present disclosure has been made in an effort to provide a method for preparing the bioadhesive composition.

An exemplary embodiment of the present disclosure provides a bioadhesive composition including mussel adhesive protein; and at least one of polyacrylic acid and polymethacrylic acid, in which the mussel adhesive protein is linked to the at least one of polyacrylic acid and polymethacrylic acid by an amide bond, and in which the bioadhesive composition has a three-dimensional network structure.

The mussel adhesive protein may include at least one amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO:

14 and SEQ ID NO: 15.

The mussel adhesive protein may include a catechol compound into which a tyrosine residue is converted; a catechol derivative introduced onto the surface of the mussel adhesive protein; or all of them.

The catechol compound may include at least one selected from the group consisting of DOPA (3,4-dihydroxyphenylalanine), Dopa o-quinone, TOPA (2,4,5-trihydroxyphenylalanine), Topa quinone and a derivative thereof.

The bioadhesive composition may include a first component in which the mussel adhesive protein and polyacrylic acid are amide-bonded; a second component in which the mussel adhesive protein and polymethacrylic acid are amide-bonded; or a mixture of the first component and the second component. The mussel adhesive protein to the polyacrylic acid or polymethacrylic acid may be included in a mass ratio of 1:1 to 5 in the first component or the second component.

The mussel adhesive protein to the polyacrylic acid or polymethacrylic acid may be included in a mass ratio of 1:2.5 to 3.5 in the first component or the second component.

The first component and the second component may be mixed in a mass ratio of 3:1 to 1:3.

The bioadhesive composition may be non-adhesive and non-degradable in a dry state and may be adhesive and biodegradable in a wet state.

The bioadhesive composition may be a film, single-sided or double-sided tape, or a hydrogel.

Further, another exemplary embodiment of the present disclosure provides a method for preparing a bioadhesive composition, the method including steps of: (a) preparing a mixed solution in which a solution containing mussel adhesive protein is mixed with a solution containing at least one of acrylic acid N-hydrosuccinimide ester and methacrylic anhydride; (b) dissolving a photoinitiator in the mixed solution; (c) adding at least one of an acrylic acid monomer and a methacrylic acid monomer to the mixed solution; and (d) curing the mixed solution.

Specifically, yet another exemplary embodiment of the present disclosure provides a method for preparing a bioadhesive composition, the method including steps of: (a-1) preparing a first mixed solution in which the solution containing mussel adhesive protein is mixed with the solution containing acrylic acid N-hydrosuccinimide ester and preparing a second mixed solution in which the solution containing mussel adhesive protein is mixed with the solution containing methacrylic anhydride, respectively; (b-1) dissolving a photoinitiator in each of the first mixed solution and the second mixed solution; (c-1) adding an acrylic acid monomer to the first mixed solution and adding a methacrylic acid monomer to the second mixed solution; and (d-1) mixing and curing the first mixed solution and the second mixed solution.

Still another exemplary embodiment of the present disclosure provides a method for preparing a bioadhesive composition, the method including steps of: (a-2) preparing a mixed solution in which the solution containing mussel adhesive protein and the solution containing acrylic acid N-hydrosuccinimide ester are mixed; (b-2) dissolving a photoinitiator in the mixed solution; (c-2) adding an acrylic acid monomer; and (d-2) curing the mixed solution.

Further, another exemplary embodiment of the present disclosure provides a method for preparing a bioadhesive composition, the method including steps of: (a-3) preparing a mixed solution in which the solution containing mussel adhesive protein and the solution containing methacrylic anhydride are mixed; (b-3) dissolving a photoinitiator in the mixed solution; (c-3) adding a methacrylic acid monomer; and (d-3) curing the mixed solution.

The photoinitiator may be a-ketoglutaric acid.

The mussel adhesive protein may be included in an amount of 7 to 15% by weight based on the total weight of the mixed solution.

The at least one of acrylic acid N-hydrosuccinimide ester and methacrylic anhydride may be included in an amount of 0.6 to 2.0% by weight based on the total weight of the mixed solution.

The photoinitiator may be included in an amount of 0.1 to 0.4% by weight based on the total weight of the mixed solution.

The step (d-1) is a step of mixing the first mixed solution and the second mixed solution in a mass ratio of 3:1 to 1:3.

In step (d-2), mussel adhesive protein to acrylic acid monomer may be included in a mass ratio of 1:1 to 5.

According to the exemplary embodiments of the present disclosure, the bioadhesive composition is formed in a three-dimensional network structure in which mussel adhesive protein and poly(meth)acrylic acid are linked by an amide bond, is non-adhesive and non-degradable in a dry state, has excellent bioadhesive ability in a wet state and may be used as a bioadhesive tape or film that may be biodegradable by bioenzymes.

Further, according to the exemplary embodiments of the present disclosure, the decomposition time of the bioadhesive composition may be controlled by crosslinking poly(meth)acrylic to two mussel adhesive proteins and then adjusting their mixing ratio, and the bioadhesive composition has excellent biostability since the toxic (meth)acrylic acid monomer is not leached and has no or very low cytotoxicity, so it may be applied to various biomedical applications requiring bioadhesion.

Further, according to the exemplary embodiments of the present disclosure, the bioadhesive composition may be used as a double-sided adhesive tape or film, may be used as a biomaterial-based tissue adhesive, so that time-limited electronic circuit for in vivo signal transmission and drug administration is attached thereto, achieving normalization of organs and periodic drug administration after attachment without additional procedures.

It should be understood that the effect of the present disclosure is not limited to the above effect but includes all effects that may be inferred from the detailed description of the present disclosure, or the configuration of the invention described in the claims.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic view of the first component in which mussel adhesive protein and polyacrylic acid are amide-bonded according to Example 1 of the present disclosure;

FIG. 2 is a schematic view of the second component of the mussel adhesive protein and polymethacrylic acid are amide-bonded according to Example 2 of the present disclosure;

FIG. 3A is a view showing the MALDI mass spectrum of the conventional mussel adhesive protein (FP-151), and FIG. 3B is a view showing the MALDI mass spectrum of the methacrylated mussel adhesive protein prepared in Example 2;

FIG. 4A is a view showing FT-IR data of the conventional mussel adhesive protein (FP-151), FIG. 4B is a view showing FT-IR data of the methacrylated mussel adhesive protein prepared in Example 2, and FIG. 4C is a view showing the result of confirming whether the composition of Example 7 was synthesized through FT-IR;

FIG. 5 is a result of confirming the decomposition data of the bioadhesive composition prepared according to Examples 1 to 3 of the present disclosure;

FIG. 6 is a result confirming the adhesive strength of the bioadhesive composition prepared according to Examples 1 to 3 of the present disclosure;

FIG. 7A, FIG. 7B, FIG. 7C and FIG. 7D are a result of evaluating the cytotoxicity of the bioadhesive composition according to the present disclosure in which

FIG. 7A is a graph showing the CCK-8 test result obtained by measuring the absorbance of Example 3 at 450 nm using a microplate absorption spectrophotometer, FIG. 7B is the result of confirming the survival of cells in Example 3 by performing the Live-dead assay (living cells are green, and dead cells are red), FIG. 7C is a graph showing the CCK-8 test result obtained by measuring the absorbance of Example 7 at 450 nm using a microplate absorption spectrophotometer, and FIG. 7D is the result of confirming the survival of cells in Example 7 by performing the Live-dead assay (living cells are green, and dead cells are red);

FIG. 8A is an image confirming the dry state of the bioadhesive composition prepared according to Examples 1 to 5 of the present disclosure, and FIG. 8B is an image confirming the dry state of the composition prepared according to Examples 6 to 9, FIG. 8C is an image showing comparing the dry state of the compositions prepared according to Comparative Examples 1, 2 and Example 7;

FIG. 9 is a result of evaluating the biodegradability of the bioadhesive composition according to the present disclosure in the absence and presence of a bioenzyme;

FIG. 10 is a result of confirming the underwater adhesion ability in pig skin using the bioadhesive composition of Example 7 according to the present disclosure, the composition of Comparative Example 2, and a commercially available adhesive;

FIG. 11A and FIG. 11B are a test result of tensile force of the bioadhesive composition of Example 2 according to the present disclosure in which FIG. 11A is a test result of tensile force of a tape in a dry state, and FIG. 11B is a test result of tensile force of the tape in a wet state; and

FIG. 12A and FIG. 12B are a result of confirming the tensile force test of the bioadhesive composition of Example 7 according to the present disclosure through repeated contraction/expansion tests in a pig bladder, in which FIG. 12A is a result of a commercially available anti-adhesion film, and FIG. 12B is a result of the composition for bioadhesiveness of Example 7.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

In the following description, only the parts necessary for understanding the example of the present disclosure are described, and it should be noted that the description of other parts may be excluded to the extent that the gist of the present disclosure is not disturbed.

The terms or words used in the present specification and claims described below should not be construed as being limited to their ordinary or dictionary meanings, and it must be interpreted as meaning and concept consistent with the technical idea of the present disclosure based on the principle that the inventor may appropriately define the concept of a term to best describe his invention. Therefore, it should be understood that the configuration shown in the examples and drawings described in this specification is only a preferred embodiment of the present disclosure and does not represent all of the technical idea of the present disclosure, and various equivalents and modifications may be substituted for them at the time of filing the present application.

Hereinafter, the present disclosure is described in detail.

The present disclosure provides a bioadhesive composition including mussel adhesive protein; and at least one of polyacrylic acid and polymethacrylic acid, in which the mussel adhesive protein is connected to at least one of polyacrylic acid and polymethacrylic acid by an amide bond and has a three-dimensional network structure.

The mussel adhesive protein is a protein derived from the mussel byssus, preferably mussel adhesive protein derived from Mytilus edulis, Mytilus galloprovincialis, or Mytilus coruscus or a mutant thereof, but is not limited thereto.

The mussel adhesive protein of the present disclosure may include Mefp (Mytilus edulis foot protein)-1, Mgfp (Mytilus galloprovincialis foot protein)-1, Mcfp (Mytilus coruscus foot protein)-1, Mefp-2, Mefp-3, Mgfp-3 and Mgfp-5 or a mutant thereof, derived from each of the above mussel species, preferably a protein selected from the group consisting of fp (foot protein)-1 (SEQ ID NO: 1), fp-2 (SEQ ID NO: 4), fp-3 (SEQ ID NO: 5), fp-4 (SEQ ID NO: 6), fp-5 (SEQ ID NO: 7), and fp-6 (SEQ ID NO: 8), a fusion protein in which two or more proteins are linked, or mutants of the above proteins, but are not limited thereto.

Further, the mussel adhesive protein of the present disclosure includes all mussel adhesive proteins described in International Publication NO. WO2006/107183 or WO2005/092920. Preferably, the mussel adhesive protein may include a fusion protein selected from the group consisting of fp-151 (SEQ ID NO: 9), fp-131 (SEQ ID NO: 10), fp-353 (SEQ ID NO: 11), fp-153 (SEQ ID NO: 12) and fp-351 (SEQ ID NO: 13) but is not limited thereto.

Further, the mussel adhesive protein of the present disclosure may include a polypeptide in which a decapeptide (SEQ ID NO: 2) repeated about 80 times is continuously linked 1 to 12 times or more in fp-1. Preferably, it may be an fp-1 variant polypeptide (SEQ ID NO: 3) in which the decapeptide represented by SEQ ID NO: 2 is continuously linked 12 times but is not limited thereto.

Further, the mussel adhesive protein of the present disclosure may be a mutant (SEQ ID NO: 15) of fp-151 but is not limited thereto. The protein sequence represented by SEQ ID NO: 15 is a sequence in which a linker sequence and the like are excluded as compared to SEQ ID NO: 9. Specifically, it is a fusion protein sequence in which the sequence of Mgfp-5 represented by SEQ ID NO: 16 is fused between the fp-1 mutant sequences represented by SEQ ID NO: 14. More specifically, the mussel adhesive protein may include at least one amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15 and SEQ ID NO: 16.

Further, in the present disclosure, the mussel adhesive protein may be modified to the extent that it contains a conservative amino acid sequence capable of maintaining the properties of the above-mentioned mussel adhesive proteins. That is, an amino acid sequence having 70% or more, preferably 80% or more, even more preferably 90% or more, in other words, 95%, 96%, 97%, 98%, 99% or more sequence identity with the amino acid sequence of the SEQ ID NOs showing substantially equivalent effects may also be included in the scope of the present disclosure.

The mussel adhesive protein may include a catechol compound into which a tyrosine residue is converted; a catechol derivative introduced onto the surface of the mussel adhesive protein; or all of them.

In the mussel adhesive protein of the present disclosure, a tyrosine residue is preferably converted into a catechol compound, and 10 to 100% of the total tyrosine residue is preferably converted into a catechol compound. In the total amino acid sequence of most mussel adhesive proteins, the proportion of tyrosine may be about 1 to 50%. Tyrosine in the mussel adhesive protein may be converted into DOPA, a catechol compound, by adding an OH group through hydration

However, since tyrosine residues are not converted in the mussel adhesive protein produced in Escherichia coli, it is preferable to perform a fertilization reaction for converting tyrosine into DOPA by a separate enzymatic and chemical treatment method. A method for modifying a tyrosine residue included in a mussel adhesive protein with a DOPA may be a method known in the art and is not particularly limited.

The catechol compound is a compound containing a dihydroxy group, and refers to a compound that imparts adhesion to a mussel adhesive protein through a crosslinking action. It specifically may include at least one selected from the group consisting of DOPA (3,4-dihydroxyphenylalanine), Dopa o-quinone, TOPA (2,4,5-trihydroxyphenylalanine), Topa quinone and a derivative thereof.

In the present disclosure, mutants of the mussel adhesive protein preferably include an additional sequence at the carboxyl or amino terminus of the mussel adhesive protein, or some amino acids are converted to other amino acids under the premise of maintaining the adhesion of the mussel adhesive protein. More preferably, a polypeptide consisting of 3 to 25 amino acids including RGD is linked to the carboxyl or amino terminus of the mussel adhesive protein, or 1 to 100%, preferably 5 to 100% of the total number of tyrosine residues constituting the mussel adhesive protein may be converted to 3,4-dihydroxyphenyl-L-alanine (DOPA).

The mussel adhesive protein may be preferably mass-produced by a genetic engineering method by inserting a foreign gene into a conventional vector constructed for the purpose of expressing the foreign gene, but it is not limited thereto. The vector may be properly selected according to the type and characteristic of the host cells or constructed de novo to produce the protein. A method of transforming host cells with the vector, and a method of producing a recombinant protein from the transformant may be easily performed using conventional methods. Methods such as selection and construction of the aforementioned vector, transformation with the vector, and expression of a recombinant protein may be easily performed by those skilled in the art, and some modifications made to the conventional methods are also encompassed in the present disclosure.

The bioadhesive composition according to the present disclosure includes mussel adhesive protein; and at least one of polyacrylic acid and polymethacrylic acid, and specifically may be a first component in which the mussel adhesive protein and polyacrylic acid are amide-bonded, or a second component in which the mussel adhesive protein and polymethacrylic acid are amide-bonded, or a mixture of the first component and the second component.

In the first component or the second component, mussel adhesive protein to polyacrylic acid or polymethacrylic acid may be included in a mass ratio of 1:1 to 5, preferably in a mass ratio of 1:2.5 to 3.5, and even more preferably in a mass ratio of 1:3. The physical properties of the prepared composition may be controlled by adjusting the content ratio (mass ratio) of the mussel adhesive protein to polyacrylic acid or polymethacrylic acid.

When the content of polyacrylic acid or polymethacrylic acid with respect to the mussel adhesive protein in the bioadhesive composition of the present disclosure is less than the above range, the protein crystals remain in the composition. When manufactured into films and tapes, the flexibility of the film is reduced, and for example, it may be broken or cracked, and expensive proteins are discarded unreacted so that economic efficiency is lowered. On the other hand, when the content of polyacrylic acid or polymethacrylic acid exceeds the above range, the ratio of polyacrylic acid or polymethacrylic acid in the composition is too high, which may inhibit the underwater adhesion of the dopamine residue of the mussel adhesive protein, and the adhesion may be reduced.

Meanwhile, when the content of polyacrylic acid or polymethacrylic acid is included in the above range, unreacted protein does not exist in the composition, so it may be prepared into films and tapes having excellent flexibility and tensile strength of the composition, so that it is possible to prepare a composition with maximization of adhesion in water by the dopamine residue of the mussel adhesive protein, excellent biodegradability and no or very low cytotoxicity.

In the bioadhesive composition of the present disclosure, the first component or the second component may be included independently, for example, the first component: the second component in a mass ratio of 10:0 or 0:10.

Further, the bioadhesive composition of the present disclosure may be a mixture of the first component and the second component, and when the first component and the second component are mixed, the first component and the second component may be mixed and used in a mass ratio of 9:1 to 1:9, preferably a mass ratio of 3:1 to 1:3, and even more preferably a mass ratio of 1:1. When the first component and the second component are mixed, the bioadhesive composition of the present disclosure adjusts the mixing ratio (mass ratio) of the first component and the second component to prepare a film or tape, etc., thereby adjusting decomposition time during adhesion in the living body. Further, the mussel adhesive protein is maximized within the mixing ratio so that it is not crystallized, and thus the protein may not be wasted.

The bioadhesive composition of a three-dimensional network structure bonded with an amide of mussel adhesive protein and at least one of polyacrylic acid and polymethacrylic acid according to the present disclosure does not exhibit adhesive force and is non-degradable in a dry state but may have adhesiveness and biodegradability in a wet state. In the wet state, the van der Waals force and the hydrophilic attraction between the mussel adhesive protein and polyacryl or polymethacrylic in the composition, and the catechol residue of the mussel adhesive protein, such as the underwater adhesive force of the dopamine residue, work together to improve the adhesion.

Further, the bioadhesive composition of a three-dimensional network structure bonded with an amide of mussel adhesive protein and at least one of polyacrylic acid and polymethacrylic acid according to the present disclosure may be biodegraded by a bioenzyme in a living body. Even when the bioadhesive composition according to the present disclosure is biodegraded, it has excellent stability because the acrylic monomer is not separated or eluted from the composition, thus there is no cytotoxicity.

The bioadhesive composition according to the present disclosure may be in the form of a film, single-sided or double-sided tape or hydrogel, but is not limited thereto, and may be usefully applied to biomedical applications requiring bioadhesion. For example, it may be used as a biomaterial-based tissue adhesive and may be applied as a double-sided adhesive tape for fixing a device mounted in a living body for in vivo signal transmission and drug administration in a living body.

Further, the present disclosure provides a method for preparing a bioadhesive composition, the method including steps of: (a) preparing a mixed solution in which a solution containing mussel adhesive protein is mixed with a solution containing at least one of acrylic acid N-hydrosuccinimide ester and methacrylic anhydride; (b) dissolving a photoinitiator in the mixed solution; (c) adding at least one of an acrylic acid monomer and a methacrylic acid monomer to the mixed solution; and (d) curing the mixed solution.

Specifically, the method for preparing a bioadhesive composition of the present disclosure may include steps of: (a-1) preparing a first mixed solution in which the solution containing mussel adhesive protein is mixed with the solution containing acrylic acid N-hydrosuccinimide ester and preparing a second mixed solution in which the solution containing mussel adhesive protein is mixed with the solution containing methacrylic anhydride, respectively; (b-1) dissolving a photoinitiator in each of the first mixed solution and the second mixed solution; (c-1) adding an acrylic acid monomer to the first mixed solution and adding a methacrylic acid monomer to the second mixed solution; and (d-1) mixing and curing the first mixed solution and the second mixed solution.

As another embodiment, the present disclosure provides a method for preparing a bioadhesive composition, the method including steps of: (a-2) preparing a mixed solution in which the solution containing mussel adhesive protein and the solution containing acrylic acid N-hydrosuccinimide ester are mixed; (b-2) dissolving a photoinitiator in the mixed solution; (c-2) adding an acrylic acid monomer; and (d-2) curing the mixed solution.

As yet another embodiment, the present disclosure provides a method for preparing a bioadhesive composition, the method including steps of: (a-3) preparing a mixed solution in which the solution containing mussel adhesive protein and the solution containing methacrylic anhydride are mixed; (b-3) dissolving a photoinitiator in the mixed solution; (c-3) adding a methacrylic acid monomer; and (d-3) curing the mixed solution.

In the present disclosure, the mussel adhesive protein is as defined above.

In the present disclosure, the acrylic acid N-hydrosuccinimide ester may be a compound represented by chemical formula 1 below, and the methacrylic anhydride may be a compound represented by chemical formula 2 below.

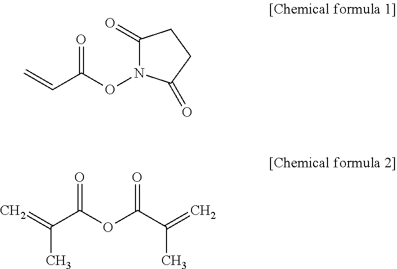

In the present disclosure, the photoinitiator may be α-ketoglutaric acid, but is not limited thereto, as long as it may form polyacrylic acid or polymethacrylic acid.

In the present disclosure, a-ketoglutaric acid may be a compound represented by chemical formula 3 below.

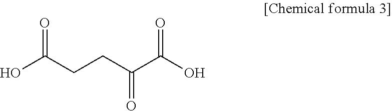

According to the present disclosure, the acrylic acid monomer may be a compound represented by the following chemical formula 4, and the methacrylic acid monomer may be a compound represented by the following chemical formula 5.

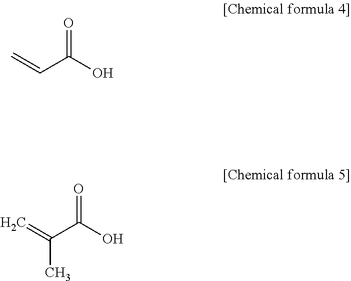

According to the present disclosure, the curing of step (d), step (d-1), step (d-2), and step (d-3) may be photocuring, and preferably it may be irradiated with 200 to 300 nm light, For example, 254 nm light. By the light irradiation and the photoinitiator, polyacrylic acid is formed from the acrylic acid monomer or polymethacrylic acid is formed from the methacrylic acid monomer, and an amide bond is formed between the mussel adhesive protein and polyacrylic acid or polymethacrylic acid.

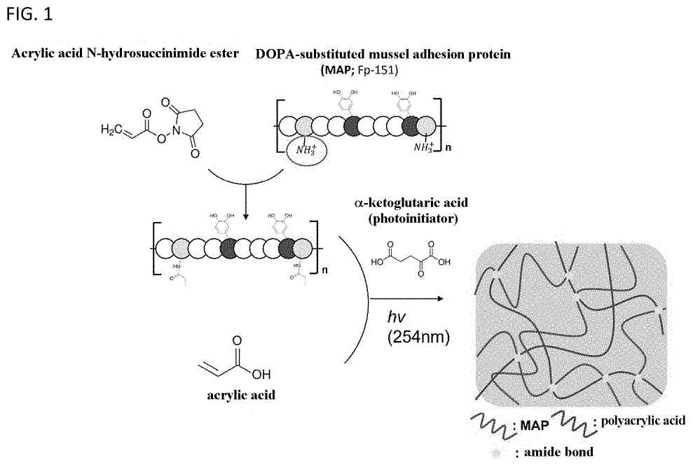

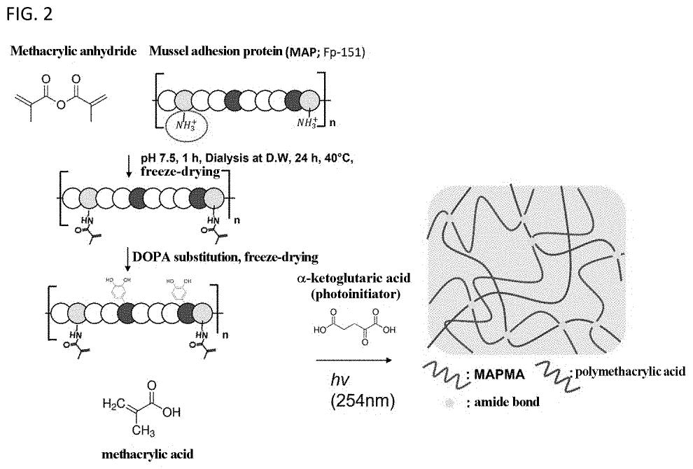

The mussel adhesive protein and the at least one polyacrylic acid and polymethacrylic acid according to the present disclosure has a three-dimensional network structure through an amide bond, which is schematically shown in FIGS. 1 and 2.

Specifically, FIG. 1 is a schematic diagram of a first component in which mussel adhesive protein and polyacrylic acid are amide-bonded, and FIG. 2 is a schematic diagram of a second component in which mussel adhesive protein and polymethacrylic acid are amide-bonded.

According to the present disclosure, the mussel adhesive protein may be included in an amount of 7 to 15% by weight based on the total weight of the mixed solution, and the mussel adhesive protein and the acrylic acid monomer or methacrylic acid monomer may be included in a mass ratio of 1:1 to 5, preferably a mass ratio of 1:2.5 to 3.5, more preferably a mass ratio of 1:3.

When the mussel adhesive protein is included in the above range based on the total weight of the mixed solution, the bioadhesiveness is excellent. In addition, when the ratio of the mussel adhesive protein to the acrylic acid monomer or methacrylic acid monomer is within the above range, the film or double-sided tape for bioadhesive which has excellent mechanical properties, such as excellent tensile force, and excellent bioadhesive ability in both dry and wet conditions may be prepared.

In the present disclosure, the photoinitiator may be included in an amount of 0.1 to 0.4 wt %, more preferably 0.2 to 0.3 wt %, based on the total weight of the mixed solution.

The photoinitiator serves as a catalyst during the reaction, and when it is included in the above range based on the total weight of the mixed solution, the reaction may be easily performed between the mussel adhesive protein and acrylic acid N-hydrosuccinimide ester or methacrylic anhydride.

In the present disclosure, the at least one of acrylic acid N-hydrosuccinimide ester and methacrylic anhydride may be included in an amount of 0.6 to 2.0% by weight, more preferably 0.8 to 1.5% by weight based on the total weight of the mixed solution. When the content of the at least one of acrylic acid N-hydrosuccinimide ester and methacrylic anhydride is less than the above range, the three-dimensional network structure is not dense, and the pore size is large so that film or tape with a reduced tensile may be prepared. When the content exceeds the above range, the three-dimensional network structure is too dense so that a film or tape may be manufactured with a lowered adhesive force as well as a low elastic force. On the other hand, when the content is included in the above range, it is particularly preferable because the tensile force is excellent, and the adhesive force is particularly improved.

In the present disclosure, the first mixed solution or the second mixed solution may be separately prepared and used, and in this case, the first mixed solution: the second mixed solution may be used in a mass ratio of 10:0 or 0:10.

Further, in the present disclosure, the first mixed solution and the second mixed solution may be mixed and used in a mass ratio of 9:1 to 1:9, preferably 3:1 to 1:3, more preferably 1:1. In this case, the bioadhesive composition of the present disclosure may be prepared as a film or tape by adjusting the mixing ratio (mass ratio) of the first mixed solution and the second mixed solution, so that the decomposition time may be adjusted during adhesion in the living body. Further, the mussel adhesive protein is maximized within the mixing ratio so that it is not crystallized, and thus the protein may not be wasted.

The method for preparing the bioadhesive composition of the present disclosure may further include step of drying after curing the mixed solution in step (d), or after mixing and curing the first mixed solution and the second mixed solution as in step (d-1), (d-2) or (d-3).

The bioadhesive composition according to the present disclosure is non-adhesive and non-degradable in a dry state and exhibits adhesiveness in a wet state. In addition, it may exhibit biodegradability under wet conditions containing bioenzymes, such as wet conditions in the presence of collagenase. However, in the biodegradation, the mussel adhesive protein binding portion is biodegraded, and toxic acrylic acid monomer or methacrylic acid monomer is not discharged so that there is no cytotoxicity, and biocompatibility is maintained.

The bioadhesive composition of the present disclosure is a wide range of promising adhesive materials and may be made of a film, single-sided or double-sided adhesive tape or hydrogel, but is not limited thereto.

Further, the bioadhesive composition may be usefully used as an adhesive for wearable electronics, artificial skin, robotics of supercapacitors, energy storage materials, bioelectrodes, implantable amperometric biosensors, electrical stimulation drug release devices, and neural prostheses.

The above description is a description of the technical idea of the present disclosure using one embodiment, and a person having ordinary knowledge in the technical field to which the present disclosure belongs may make various modifications and variations within the scope without departing from the essential characteristics of the present disclosure. Therefore, the examples described in the present disclosure is for explaining rather than limiting the technical idea of the present disclosure, and the scope of the technical idea of the present disclosure is not limited by these examples. The protection scope of the present disclosure should be interpreted by the claims, and all technical ideas within the scope equivalent thereto should be construed as being included in the scope of the present disclosure.

### PREPARATION EXAMPLES

### Preparation Example 1: Preparation of Mussel Adhesive Protein

A mussel adhesive protein (fp-151) represented by SEQ ID NO: 9 was prepared as follows. An fp-1 mutant consisting of six repeats of decapeptide was synthesized, in order to express the decapeptide in E. coli, in which the decapeptide consists of 10 amino acids that is repeated about 80 times in a natural mussel adhesive protein fp-1. The Mgfp-5 gene (Genbank No. AAS00463 or AY521220) was inserted between two fp-1 mutants and expressed in E. coli to produce fp-151 (D. S. Hwang et. al., Biomaterials 28, 3560-3568, 2007). Next, tyrosine of the mussel adhesive protein was converted into DOPA through an in vitro enzymatic reaction using mushroom tyrosinase to the mussel adhesive protein (fp-151) represented by SEQ ID NO: 9 to prepare a mussel adhesive protein into which a catechol DOPA residue was introduced.

### Preparation Example 2: Preparation of Mussel Adhesive Protein

A mussel adhesive protein (fp-151) represented by SEQ ID NO: 9 was prepared as follows. An fp-1 mutant consisting of six repeats of decapeptide was synthesized, in order to express the decapeptide in E. coli, in which the decapeptide consists of 10 amino acids that is repeated about 80 times in a natural mussel adhesive protein fp-1. The Mgfp-5 gene (Genbank No. AAS00463 or AY521220) was inserted between two fp-1 mutants and expressed in E. coli to produce fp-151 (D. S. Hwang et. al., Biomaterials 28, 3560-3568, 2007).

### EXAMPLES

### Example 1. Preparation of the First Component

10% by weight of the catechol DOPA residue-introduced mussel adhesive protein prepared in Preparation Example 1, 1% by weight of acrylic acid N-hydrosuccinimide ester, and 0.2% by weight of α-ketoglutaric acid as a photoinitiator were dissolved in deionized water. Next, acrylic acid was added at a concentration of 20% by weight. They were mixed to prepare the first mixed solution. The first mixed solution was filtered through a 0.4 μm sterile syringe filter and poured into a quartz mold. Next, they were cured using an ultraviolet (UV) lamp at a wavelength of 254 nm and a power of 10 W for 30 minutes.

### Example 2. Preparation of the Second Component

The mussel adhesive protein prepared in Preparation Example 2 was dissolved at a concentration of 0.1% by weight in a buffer solution (pbs of pH 7.5). Then, 2 ml of methacrylic anhydride was added per 1 g of the mussel adhesive protein. They were reacted in a water bath at 40° C. by stirring at 120 rpm for 1 hour. The mixture was put in Dialysis bag No. 4 (Spectra/Por 4 dialysis membrane Standard RC Tubing), followed by dialysis in D.W. in a water bath at 40° C. for one day. Then, the methacrylic group-introduced into mussel adhesive protein was freeze-dried, and tyrosine of mussel adhesive protein was converted into a DOPA to prepare a mussel adhesive protein into which a catechol DOPA residue was introduced. 10% by weight of the catechol DOPA residue-introduced mussel adhesive protein and 0.2% by weight of α-ketoglutaric acid as a photoinitiator was dissolved in deionized water. Next, methacrylic acid was added at a concentration of 30% by weight. They were mixed to prepare the second mixed solution. The second mixed solution was filtered through a 0.4 μm sterile syringe filter and poured into a quartz mold. Next, they were cured using an ultraviolet (UV) lamp at a wavelength of 254 nm and a power of 10 W for 1 hour.

### Example 3. Mixing of the First Component and the Second Component (in a Mass Ratio of 1:1)

A mixed solution obtained by mixing the first mixed solution prepared according to Example 1 and the second mixed solution prepared according to Example 2 in a mass ratio of 1:1 was filtered through a 0.4 μm sterile syringe filter and poured into a quartz mold. Next, they were cured using an ultraviolet (UV) lamp at a wavelength of 254 nm and a power of 10 W for 30 minutes.

### Example 4. Mixing of the First Component and the Second Component (in a Mass Ratio of 1:3)

A mixed solution obtained by mixing the first mixed solution prepared according to Example 1 and the second mixed solution prepared according to Example 2 in a mass ratio of 1:3 was filtered through a 0.4 μm sterile syringe filter and poured into a quartz mold. Next, they were cured using an ultraviolet (UV) lamp at a wavelength of 254 nm and a power of 10 W for 30 minutes.

### Example 5. Mixing of the First Component and the Second Component (in a Mass Ratio of 3:1)

A mixed solution obtained by mixing the first mixed solution prepared according to Example 1 and the second mixed solution prepared according to Example 2 in a mass ratio of 3:1 was filtered through a 0.4 μm sterile syringe filter and poured into a quartz mold. Next, they were cured using an ultraviolet (UV) lamp at a wavelength of 254 nm and a power of 10 W for 30 minutes.

### Example 6. Preparation of the First Component (Mussel Adhesive Protein: Acrylic Acid=1:2 in a Mass Ratio)

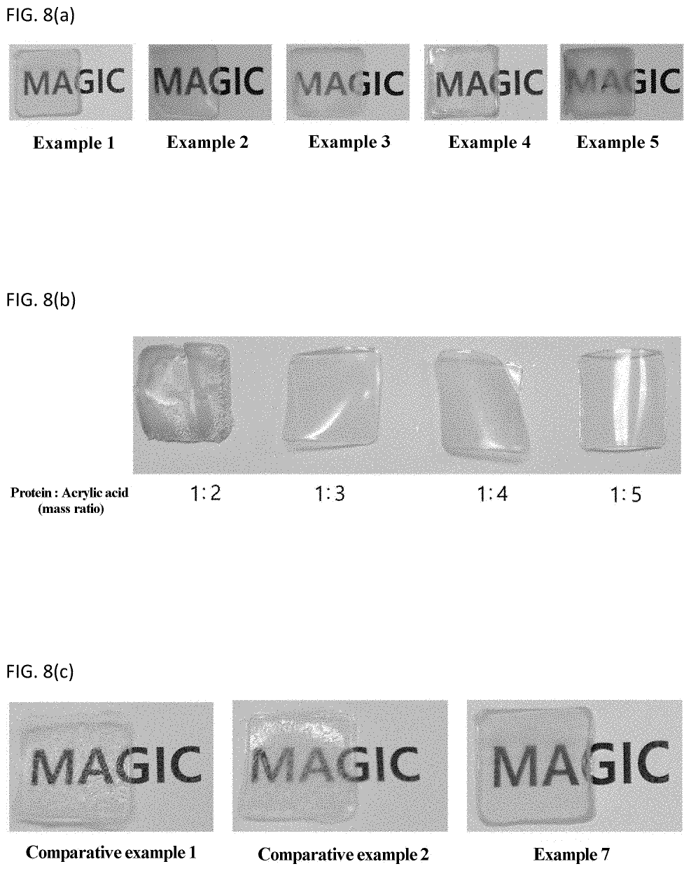

10% by weight of the catechol DOPA residue-introduced mussel adhesive protein prepared in Preparation Example 1, 1% by weight of acrylic acid N-hydrosuccinimide ester, and 0.2% by weight of α-ketoglutaric acid as a photoinitiator were dissolved in deionized water. Next, acrylic acid was added at a concentration of 20% by weight, and they were mixed. The mixed solution was filtered through a 0.4 μm sterile syringe filter and poured into a quartz mold. Next, they were cured using an ultraviolet (UV) lamp at a wavelength of 254 nm and a power of 10 W for 1 hour and were completely dried. An image of the dry state of the composition is shown in FIG. 8B.

### Example 7. Preparation of the First Component (Mussel Adhesive Protein: Acrylic Acid=1:3 in a Mass Ratio)

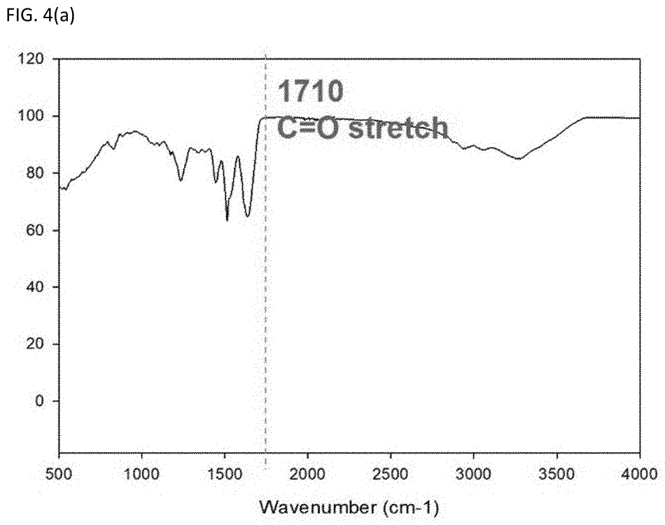

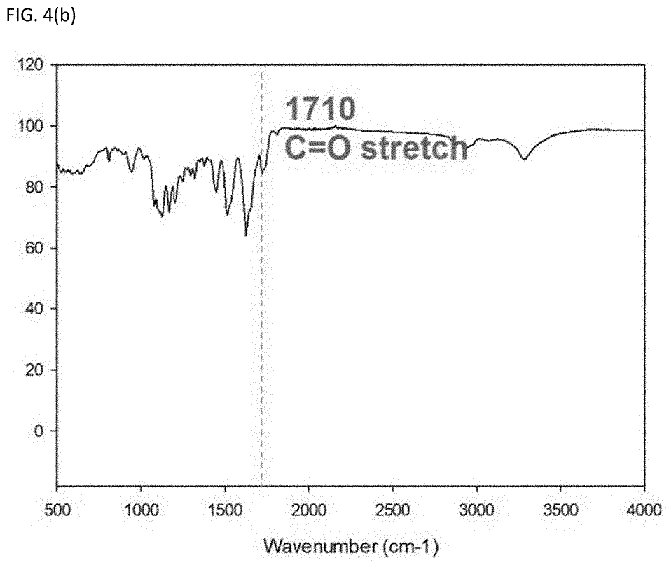

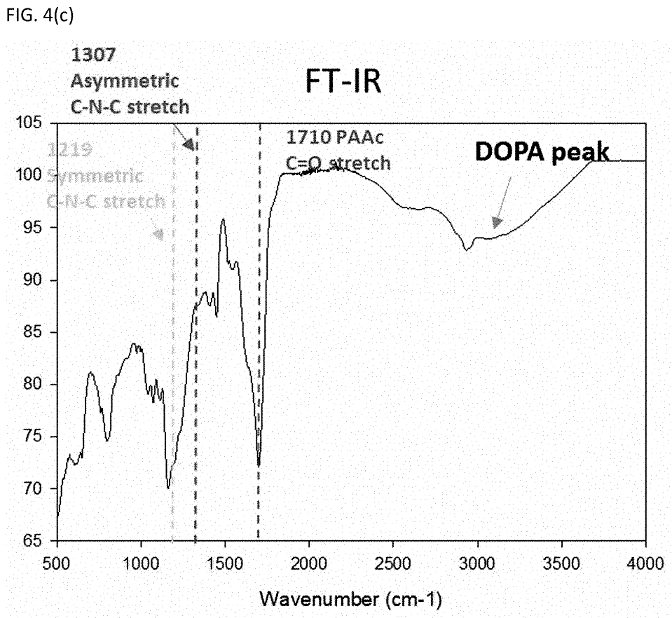

A composition in which mussel adhesive protein and polyacrylic acid were crosslinked was prepared by the method of Example 6, except that the concentration of acrylic acid was 30% by weight. Images of the dry state of the composition are shown in FIGS. 8B and 8C. The prepared composition was confirmed by FT-IR, and the results are shown in FIG. 4C. A DOPA peak appeared at 3000 cm-1, C═O stretching of polyacrylic acid was confirmed at 1710 cm-1, symmetrical C—N—C stretching was confirmed at 1219 cm-1, and asymmetrical S—N—C stretching was confirmed at 1307 cm-1, verifying that the composition was normally synthesized.

### Example 8. Preparation of the First Component (Mussel Adhesive Protein: Acrylic Acid=1:4 in a Mass Ratio)

A composition in which mussel adhesive protein and polyacrylic acid were crosslinked was prepared by the method of Example 6, except that the concentration of acrylic acid was 40% by weight. An Image of the dry state of the composition is shown in FIG. 8B.

### Example 9. Preparation of the First Component (Mussel Adhesive Protein: Acrylic Acid=1:5 in a Mass Ratio)

A composition in which mussel adhesive protein and polyacrylic acid were crosslinked was prepared by the method of Example 6, except that the concentration of acrylic acid was 50% by weight. An Image of the dry state of the composition is shown in FIG. 8B.

### COMPARATIVE EXAMPLES

### Comparative Example 1

Gelatin was added instead of mussel adhesive protein, and a composition was prepared by the method of Example 1 so that the concentration of acrylic acid contained 30% by weight. The prepared composition is shown in FIG. 8C.

### Comparative Example 2. Composition Including Unmodified Mussel Adhesive Protein and Polyacrylic Acid

A composition including an unmodified mussel adhesive protein into which the DOPA was not introduced and acrylic acid at a concentration of 30% by weight was prepared by the method of Example 6. The prepared composition is shown in FIG. 8C.

As shown in FIG. 8B, when the mass ratio of mussel adhesive protein: polyacrylic acid was 1:2, it showed somewhat weak mechanical properties in the dry state. On the other hand, it was confirmed that when the mass ratio of mussel adhesive protein: polyacrylic acid exceeded 1:4, its hardness was strong, but elasticity was low in a wet state. As a result of the experiment, the composition of Example 7 in which the mass ratio of mussel adhesive protein:polyacrylic acid was 1:3 was evaluated to have excellent mechanical properties, tensile force and elasticity.

Meanwhile, as a result of comparing Comparative Examples 1 and 2 and Example 7, the composition of Example 7 was excellent in mechanical stability in a dry state and excellent in tensile strength in a wet state compared to Comparative Examples 1 and 2.

### Test Example

### Test Example 1. MALDI Mass Spectrometry

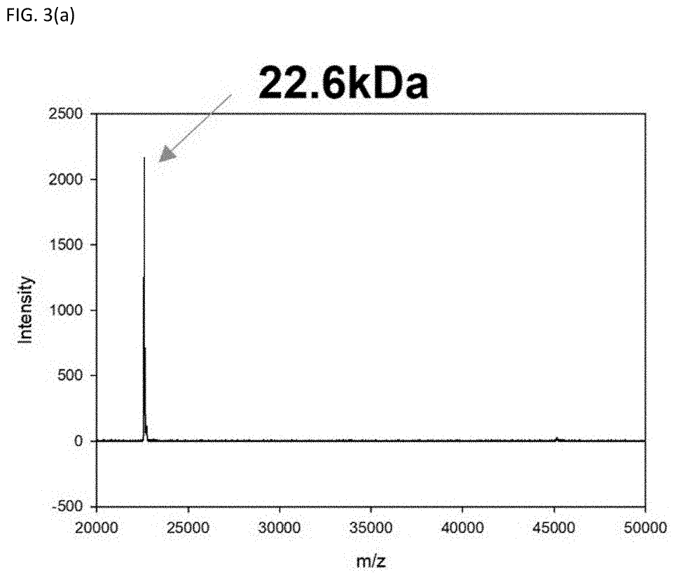

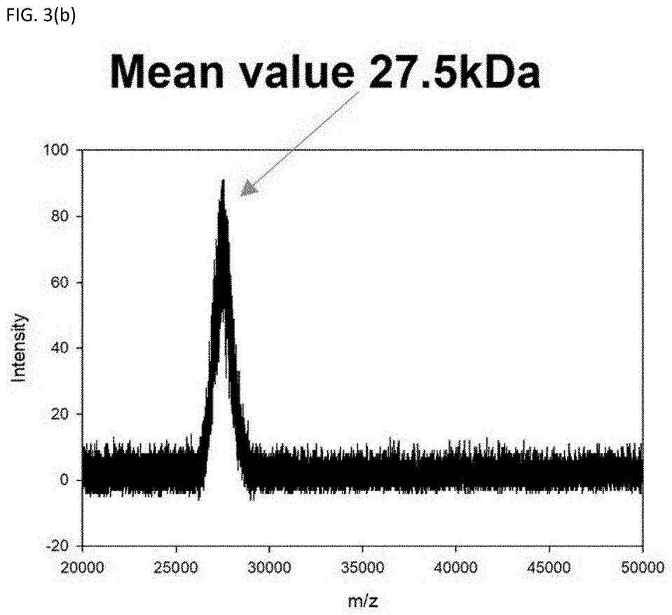

The conventional mussel adhesive protein (fp-151) and the mussel adhesive protein methacrylated by introducing a methacrylic group into the mussel adhesive protein prepared in Example 2 were crystallized and placed on a maldi plate to examine the molecular weight. The results are shown in FIGS. 3A and 3B.

FIG. 3 shows MALDI mass spectra of FIG. 3A a conventional mussel adhesive protein (FP-151) and FIG. 3B a methacrylated mussel adhesive protein prepared in Example 2.

Referring to FIGS. 3A and 3B, the average molecular weight in the large peak increased from 22.6 kDa to 27.5 kDa, indicating that the mussel adhesive protein was methacrylated.

### Test Example 2. FT-IR Analysis

The conventional mussel adhesive protein (fp-151) and the mussel adhesive protein methacrylated by introducing a methacrylic group into the mussel adhesive protein prepared in Example 2 were crystallized to measure FT-IR. The results are shown in FIG. 4.

Referring to FIG. 4, it was confirmed that the mussel adhesive protein according to Example 2 was methacrylated through a change in the peak at 1710 cm⁻¹ that was confirmed when gelatin was changed to gelatin methacrylate.

### Test Example 3. Decomposition Test

The adhesive compositions prepared according to Examples 1 to 3 were prepared to have a width of 1 cm, a length of 1 cm, and a height of 0.5 mm

The adhesive compositions prepared according to Examples 1 to 3 were put into 10 ml of a pbs solution containing 0.01% of collagenase and 0.01% of lysozyme, placed in an incubator at a temperature of 37 degrees for up to 2 weeks, and then recovered. The dried weight before putting in the pbs solution and the lyophilized weight after recovery were compared to confirm the residual amount in %. The results are shown in FIG. 5.

### Test Example 4. Adhesion Test

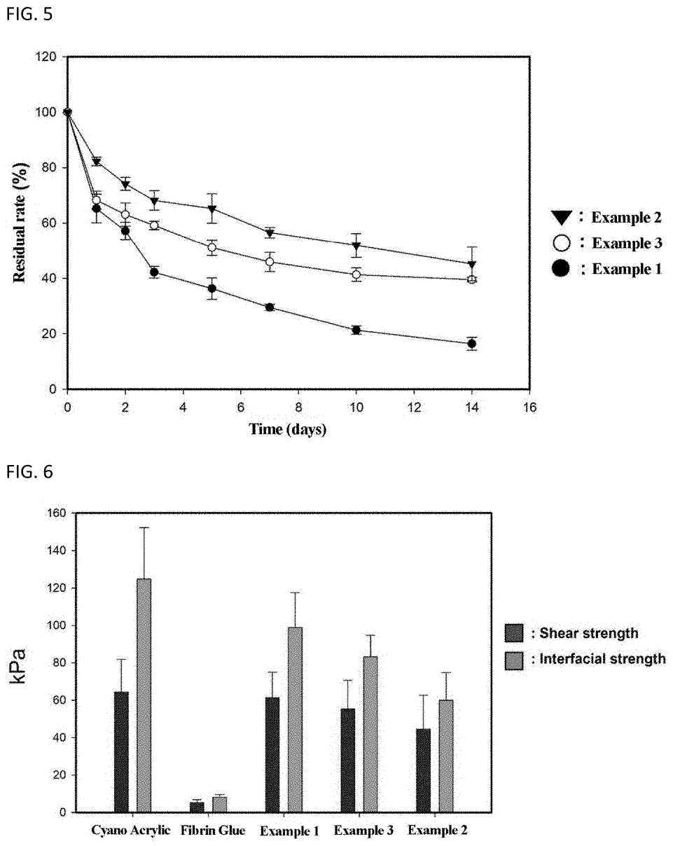

The adhesive compositions prepared according to Examples 1 to 3 and cyano acryl and fibrin glue as controls were prepared. The adhesive compositions prepared according to Examples 1 to 3 were prepared to have a width of 1 cm, a length of 1 cm, and a height of 0.5 mm, and cyano acryl and fibrin glue were used in 300 μl each. The pig skin was cut in a width of 1 cm and a length of 1 cm and attached to an acrylic plate, and an adhesive was attached between the pig skins. After they were put in pbs for 4 hours, shear stress and interfacial stress were measured. The results are shown in FIG. 6.

Referring to FIG. 6, it was confirmed that the adhesive composition according to the present disclosure had lower adhesive strength compared to cyano acryl, which is difficult to use on the human body, but showed superior adhesion compared to commercially available fibrin glue.

### Test Example 5. Cytotoxicity Test

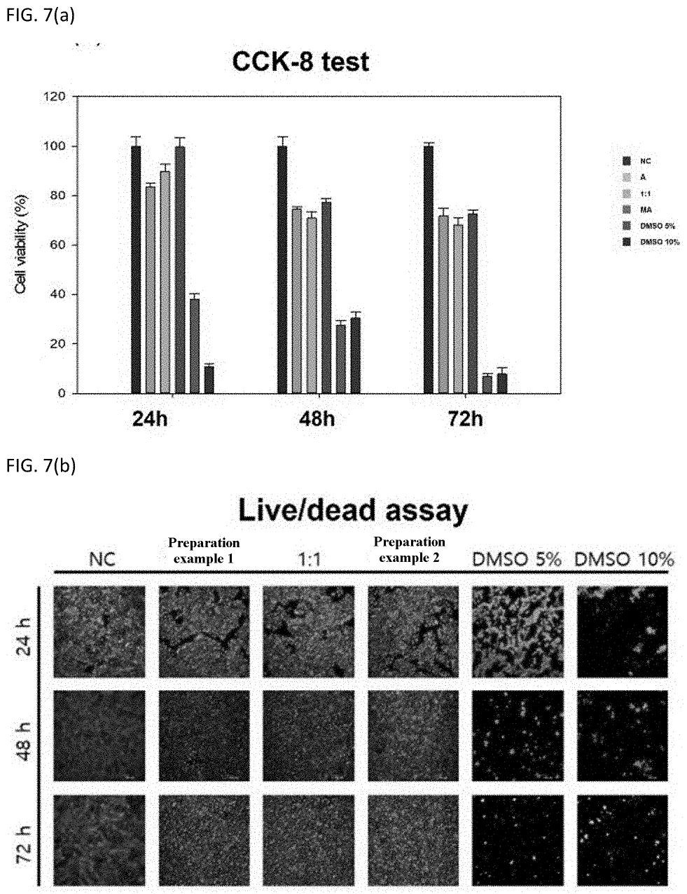

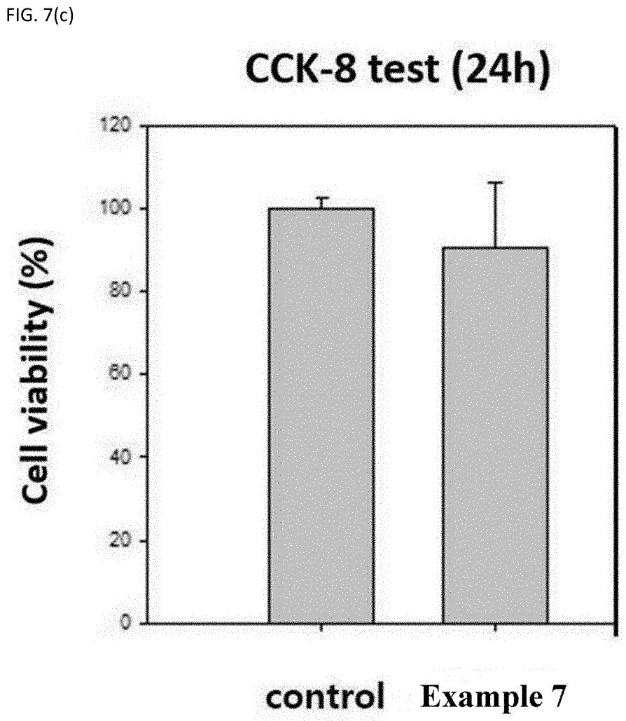

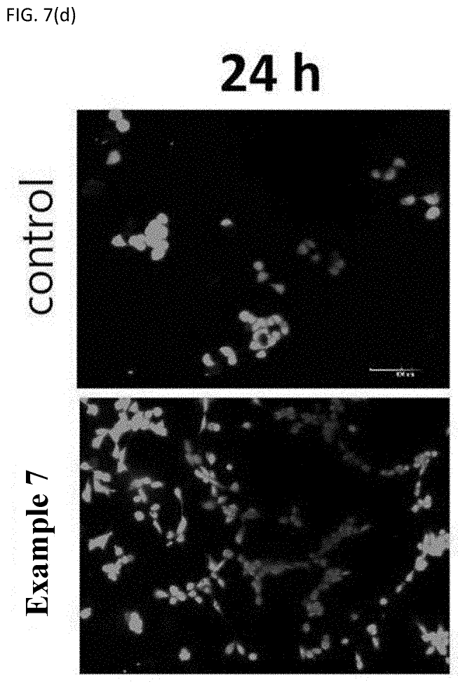

HaCaT cells were inoculated into 24-well tissue culture plates at a density of 5×10⁴ cells per well and then cultured overnight. The cells were treated with the 2% solution of Example 3 and Example 7, respectively, dissolved in Dulbecco Modified Eagle Medium (DMEM) high glucose solution. After culturing the cells for 1 day, 5% CCK-8 (Dojindo Laboratories, Tokyo, Japan) reagent dissolved in DMEM was added to each well, and the cells were incubated for 3 hours. The absorbance was measured at 450 nm using a microplate absorbance spectrophotometer. Further, cytotoxicity was evaluated by performing a Live-dead assay under the same conditions (living cells are green, dead cells are red), which are shown in FIGS. 7A to 7D.

FIG. 7A is a graph showing the results of the CCK-8 test obtained by measuring the absorbance of Example 3 at 450 nm using a microplate absorption spectrophotometer. FIG. 7B is a result of confirming the survival of cell viability of Example 3 by performing a Live-dead assay (living cells are green, dead cells are red). FIG. 7C is a graph showing the CCK-8 test results obtained by measuring the absorbance of Example 7 at 450 nm using a microplate absorption spectrophotometer. FIG. 7D is a result of confirming the survival of cell viability of Example 7 by performing a Live-dead assay (living cells are green, dead cells are red).

As shown in FIG. 7A, the cell viability was 70% or more in the CCK-8 test. Further, as shown in FIG. 7B, almost no red was observed. These results confirm that the bioadhesive composition according to the present disclosure has very low cytotoxicity. In addition, these results suggest that leaching of the acrylic monomer does not occur.

As shown in FIG. 7C, the cell viability was 90% or more in the CCK-8 test. In addition, as shown in FIG. 7D, almost no red was observed. These results confirm that the bioadhesive composition according to the present disclosure has very low cytotoxicity. Further, these results suggest that leaching of the acrylic monomer does not occur.

### Test Example 6. Confirmation of Adhesive Formation

It was confirmed that the adhesive could be formed at various concentration changes (first component: the second component=10:0, 3:1, 1:1, 1:3, 0:10) of the first component and the second component from the bioadhesive composition prepared according to Examples 1 to 5. The results are shown FIG. 8.

### Test Example 7. Evaluation of In Vitro Biodegradability

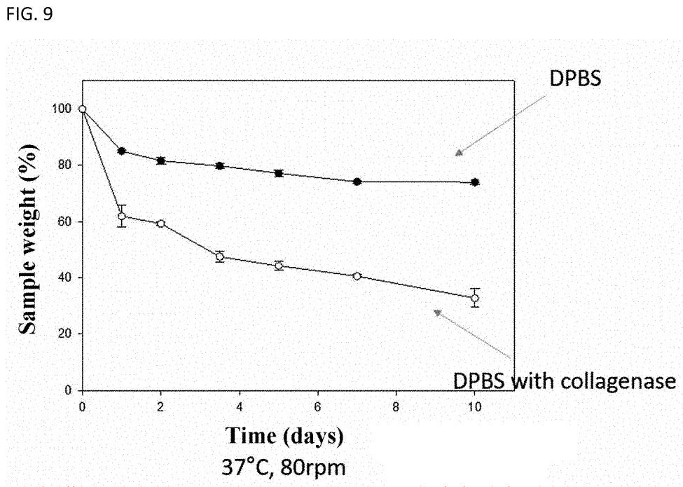

The bioadhesive composition of Example 7 was used to prepare film samples having a size of about 10 mm×10 mm having the same weight. In order to evaluate the biodegradability, two types of media were prepared to include Dulbecco's Phosphate-Buffered Saline (DPBS) containing 0.01% (w/v) sodium azide to prevent the propagation of other microorganisms in vitro, which was added with 0.005% (w/v) collagenase or without collagenase. The prepared samples were sterilized in 75% ethanol for 15 minutes before being immersed in each medium and washed three times with DPBS and then used. Each of 15 ml of a medium without collagenase or medium added with 5% collagenase was put to the conical tube, and then a sample of the bioadhesive composition was placed thereon. They were incubated at 37° C. while shaking at 60 rpm to evaluate the degree of biodegradation over time. For evaluation, the samples of the bioadhesive composition were taken out of the culture medium, washed thoroughly with deionized water, and freeze-dried. Weight loss was determined as a percentage of the mass of the freeze-dried sample after the experiment at each time interval normalized by the dry mass of the freeze-dried sample before the experiment, and the results are shown in FIG. 9.

The bioadhesive composition according to the present disclosure showed a tendency to slowly biodegrade in DPBS medium and showed a tendency to biodegrade 50% or more within 4 hours in the presence of the bioenzyme, collagenase. This means that the bioadhesive medium according to the present disclosure is easily biodegradable in the presence of enzymes, such as in vivo environments.

### Test Example 8. Underwater Adhesion Test

In order to confirm the underwater adhesion of the film, a tensile force test was performed using Instron. Before testing, the tape prepared from the bioadhesive composition of Example 7 and pig skin was covered with Dulbecco's Phosphate-Buffered Saline (DPBS) added with 0.01% (w/v) sodium azide and they were stored for 10 minutes or more to prevent degradation, dehydration and contamination. For the test, the wet pig skin was cut into 10 mm×10 mm and attached to an acrylic bar. The tape of Example 7 at 10 mm×10 mm was placed between the pig skin, and the surface was washed with PBS. The tapes of Example 7 were adhered onto the pig skin by compressing them for 5 seconds at a pressure of 1 kPa. Mechanical tests on the adhesive samples were performed for 6 hours after the initial press in order to ensure the parallel expansion of the adhesive tape in a humid environment. The shear strength and interfacial strength of two commercially available adhesives (fibrin glue, gelatin) and the composition of Comparative Example 2, as a control, were checked for each material. All tests were performed at a constant peel rate of 50 mm/min, and the interfacial toughness was determined by dividing twice the plateau force (in the case of the 180° peel test) or the plateau force (in the case of the 90° peel test).

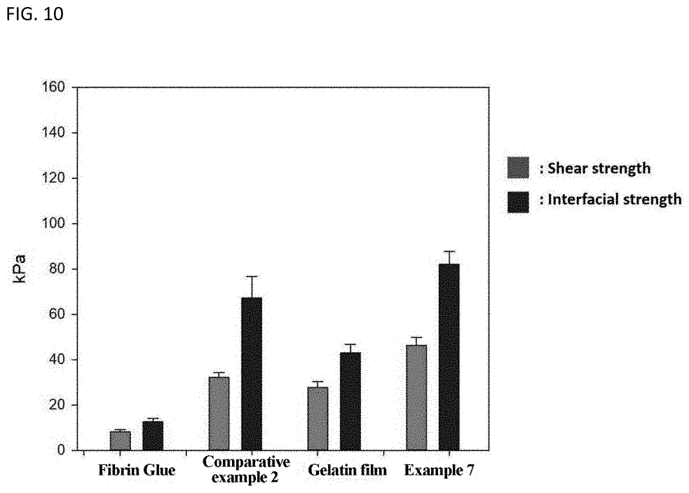

As shown in FIG. 10, the bioadhesive composition of Example 7 according to the present disclosure had superior underwater adhesion compared to commercially available adhesives, fibrin glue and gelatin. Further, it was confirmed that the modification of the surface with a catechol compound further improved the adhesion.

### Test Example 9. Tensile Force Test

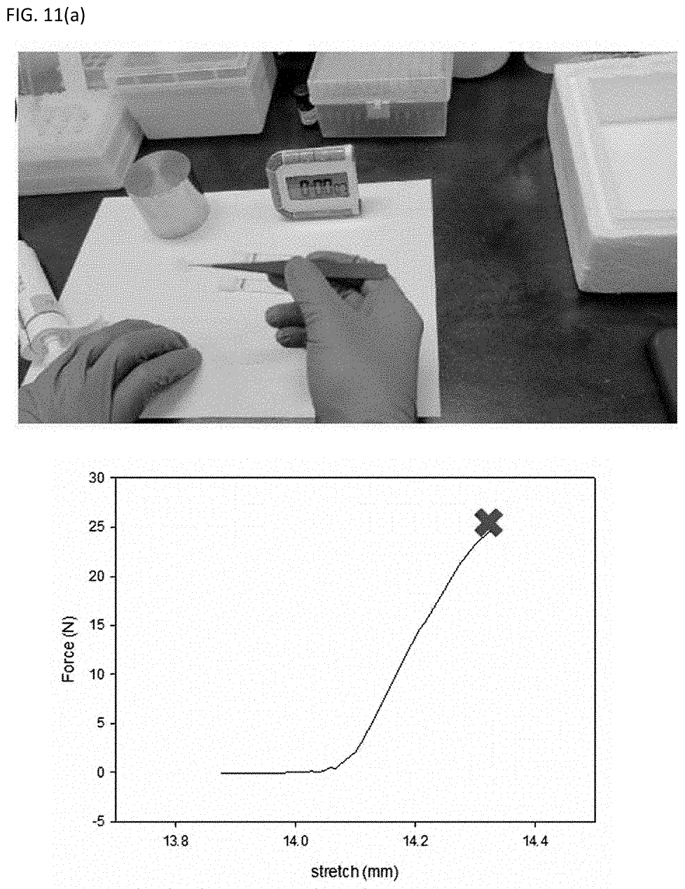

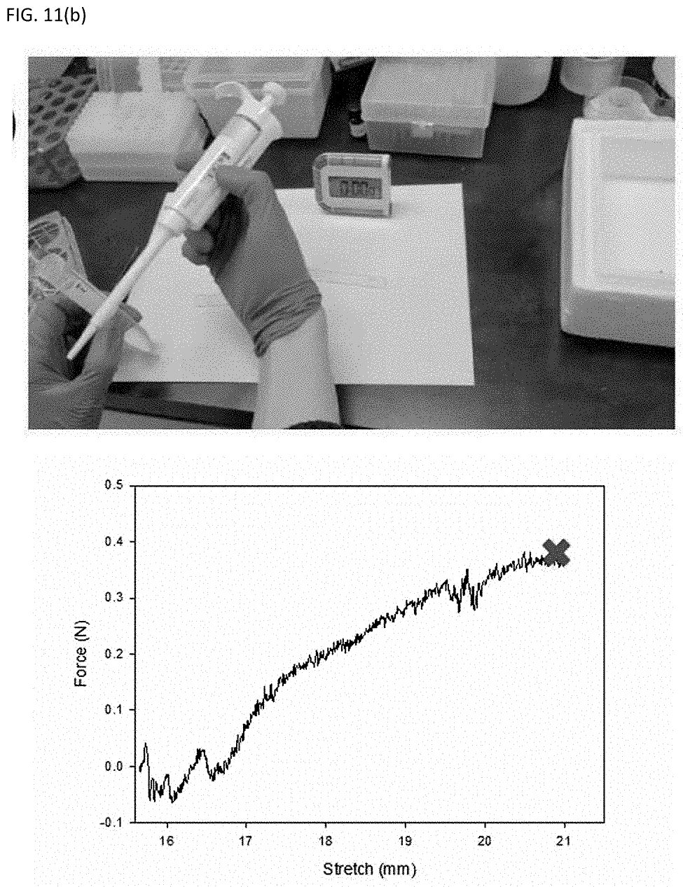

The tape of Example 7 of 1 cm×1 cm was fixed to the device and pulled up and down at a speed of 50 mm/min with an Instron. It was checked that the tape was completely dry and wet, respectively, and it was checked until the moment the tape was torn. The wet tape was prepared by completely immersing the completely dry tape in DPBS for 10 seconds and taking out the same. FIG. 11A shows a test result of a tape tensile force in a dry state, and FIG. 11B shows a test result of a tape tensile force in a wet state.

Referring to FIGS. 11A and 11B, it elongated by 0.4583 mm in the dry state, which was elongated by 4.6%. On the other hand, it was confirmed that it stretched to 5.32 mm in the wet state, which was elongated by 53.2%.

### Test Example 10. Tensile Force Test in Pig Bladder

After attaching the extracted pig bladder with the film of the bioadhesive composition of Example 7 of the present disclosure and the commercially available anti-adhesion film as a control, 250 ml of PBS was poured into the pig bladder at an injection rate of 2.5 ml/s in a water tank filled with 1×PBS. While the injection was repeated 9 times, the performance change of the attached film was observed.

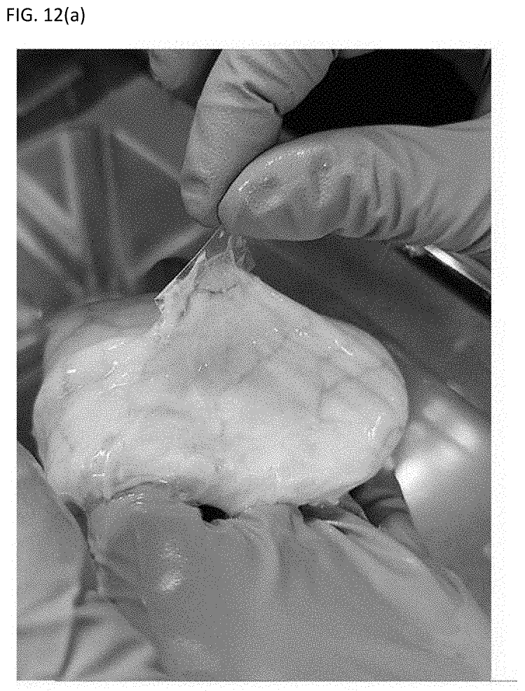

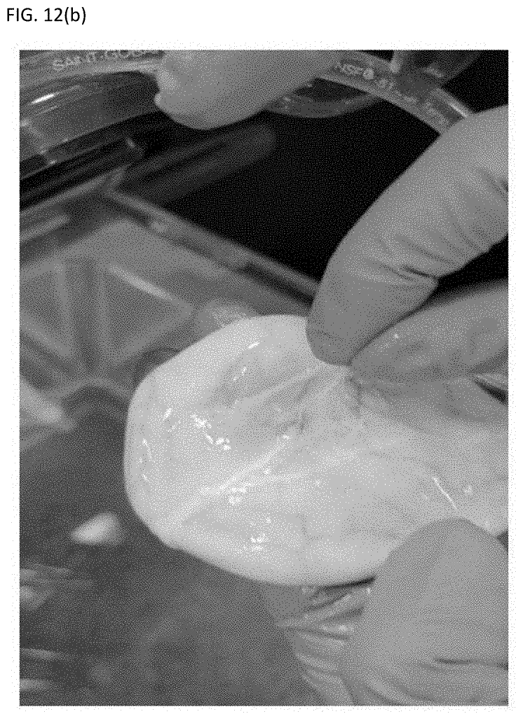

FIG. 12A shows an image of a pig's bladder to which an anti-adhesion film is attached, and FIG. 12B shows an image of a pig's bladder to which the film of Example 7 is attached. FIG. 12A shows the result of the adhesion part falling from the bladder while the anti-adhesion film does not stretch as much as the bladder in the repeated bladder expansion/contraction test. On the other hand, FIG. 12B shows that the film of Example 7 showed a tendency to expand/contract together even in repeated bladder expansion/contraction tests, and the adhesion to the surface of the pig bladder was maintained with excellent efficiency.

It will be appreciated by those skilled in the art or those having ordinary knowledge in the technical field that although described with reference to a preferred example of the present disclosure, the present disclosure may be variously modified and changed within the scope without departing from the spirit and technical scope of the present disclosure described in the claims.

Accordingly, the technical scope of the present disclosure should not be limited to the content described in the detailed description of the specification but should be defined by the claims.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A bioadhesive composition comprising mussel adhesive protein; and at least one of polyacrylic acid and polymethacrylic acid;
wherein the mussel adhesive protein is linked to the at least one of polyacrylic acid and polymethacrylic acid by an amide bond, and
wherein the bioadhesive composition has a three-dimensional network structure.

2. The bioadhesive composition of claim 1, wherein the mussel adhesive protein includes at least one amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15.

3. The bioadhesive composition of claim 1, wherein the mussel adhesive protein includes a catechol compound into which a tyrosine residue is converted; a catechol derivative introduced onto the surface of the mussel adhesive protein; or all of them.

4. The bioadhesive composition of claim 3, wherein the catechol compound includes at least one selected from the group consisting of DOPA (3,4-dihydroxyphenylalanine), Dopa o-quinone, TOPA (2,4,5-trihydroxyphenylalanine), Topa quinone and a derivative thereof.

5. The bioadhesive composition of claim 1, wherein the bioadhesive composition includes:
a first component in which the mussel adhesive protein and polyacrylic acid are amide-bonded;
a second component in which the mussel adhesive protein and polymethacrylic acid are amide-bonded; or
a mixture of the first component and the second component.

6. The bioadhesive composition of claim 5, wherein the mussel adhesive protein to the polyacrylic acid or polymethacrylic acid is included in a mass ratio of 1:1 to 5 in the first component or the second component.

7. The bioadhesive composition of claim 6, wherein the mussel adhesive protein to the polyacrylic acid or polymethacrylic acid is included in a mass ratio of 1:2.5 to 3.5 in the first component or the second component.

8. The bioadhesive composition of claim 5, wherein the first component and the second component is mixed in a mass ratio of 3:1 to 1:3.

9. The bioadhesive composition of claim 1, wherein the bioadhesive composition is non-adhesive and non-degradable in a dry state and is adhesive and biodegradable in a wet state.

10. The bioadhesive composition of claim 1, wherein the bioadhesive composition is a film, single-sided or double-sided tape, or a hydrogel.

11. A method for preparing a bioadhesive composition, the method comprising steps of:
(a) preparing a mixed solution in which a solution containing mussel adhesive protein is mixed with a solution containing at least one of acrylic acid N-hydrosuccinimide ester and methacrylic anhydride;
(b) dissolving a photoinitiator in the mixed solution;
(c) adding at least one of an acrylic acid monomer and a methacrylic acid monomer to the mixed solution; and
(d) curing the mixed solution.

12. The method of claim 11, comprising steps of:
(a-1) preparing a first mixed solution in which the solution containing mussel adhesive protein is mixed with the solution containing acrylic acid N-hydrosuccinimide ester and preparing a second mixed solution in which the solution containing mussel adhesive protein is mixed with the solution containing methacrylic anhydride, respectively;
(b-1) dissolving a photoinitiator in each of the first mixed solution and the second mixed solution;
(c-1) adding an acrylic acid monomer to the first mixed solution and adding a methacrylic acid monomer to the second mixed solution; and
(d-1) mixing and curing the first mixed solution and the second mixed solution.

13. The method of claim 11, wherein the photoinitiator is α-ketoglutaric acid.

14. The method of claim 11, wherein the mussel adhesive protein is included in an amount of 7 to 15% by weight based on the total weight of the mixed solution.

15. The method of claim 11, wherein the at least one of acrylic acid N-hydrosuccinimide ester and methacrylic anhydride is included in an amount of 0.6 to 2.0% by weight based on the total weight of the mixed solution.

16. The method of claim 11, wherein the photoinitiator is included in an amount of 0.1 to 0.4% by weight based on the total weight of the mixed solution.

17. The method of claim 12, wherein the step (d-1) is a step of mixing the first mixed solution and the second mixed solution in a mass ratio of 3:1 to 1:3.

18. The method of claim 11, wherein the bioadhesive composition is non-adhesive and non-degradable in a dry state and is adhesive and biodegradable in a wet state.

19. The method of claim 11, wherein a tyrosine residue in the mussel adhesive protein is converted into at least one catechol compound selected from the group consisting of DOPA (3,4-dihydroxyphenylalanine), Dopa o-quinone, TOPA (2,4,5-trihydroxyphenylalanine), Topa quinone and a derivative thereof.

20. The method of claim 11, wherein the mussel adhesive protein includes at least one amino acid sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14 and SEQ ID NO: 15.


SEQUENCE LISTING (16 sequences)

SEQ ID NO: 1 - protein, 800 aa, Artificial Sequence
  fp-1
AKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYK

SEQ ID NO: 2 - protein, 10 aa, Artificial Sequence
  fp-1 variant
AKPSYPPTYK

SEQ ID NO: 3 - protein, 120 aa, Artificial Sequence
  fp-1 variant
AKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYK

SEQ ID NO: 4 - protein, 472 aa, Artificial Sequence
  fp-2
LFSFFLLLTCTQLCLGTNRPDYNDDEEDDYKPPVYKPSPSKYRPVNPCLKKPCKYNGVCKPRGGSYKCFCKGGYYGYNCNLKNACKPNQCKNKSRCVPVGKTFKCVCRNGNFGRLCEKNVCSPNPCKNNGKCSPLGKTGYKCTCSGGYTGPRCEVHACKPNPCKNKGRCFPDGKTGYKCRCVDGYSGPTCQENACKPNPCSNGGTCSADKFGDYSCECRPGYFGPECERYVCAPNPCKNGGICSSDGSGGYRCRCKGGYSGPTCKVNVCKPTPCKNSGRCVNKGSSYNCICKGGYSGPTCGENVCKPNPCQNRGRCYPDNSDDGFKCRCVGGYKGPTCEDKPNPCNTKPCKNGGKCNYNGKIYTCKCAYGWRGRHCTDKAYKPNPCVVSKPCKNRGKCIWNGKAYRCKCAYGYGGRHCTKKSYKKNPCASRPCKNRGKCTDKGNGYVCKCARGYSGRYCSLKSPPSYDDDEY

SEQ ID NO: 5 - protein, 50 aa, Artificial Sequence
  fp-3
PWADYYGPKYGPPRRYGGGNYNRYGRRYGGYKGWNNGWKRGRWGRKYYGS

SEQ ID NO: 6 - protein, 750 aa, Artificial Sequence
  fp-4
YGRRYGEPSGYANIGHRRYYERAISFHRHSHVHGHHLLHRHVHRHSVLHGHVHMHRVSHRIMHRHRVLHGHVHRHRVLHNHVHRHSVLHGHVHRHRVLHRHVHRHNVLHGHVHRHRVLHKHVHNHRVLHKHLHKHQVLHGHVHRHQVLHKHVHNHRVLHKHLHKHQVLHGHVHTHRVLHKHVHKHRVLHKHLHKHQVLHGHIHTHRVLHKHLHKHQVLHGHVHTHRVLHKHVHKHRVLHKHLHKHQVLHGHVHMHRVLHKHVHKHRVLHKHVHKHHVVHKHVHSHRVLHKHVHKHRVEHQHVHKHHVLHRHVHSHHVVHSHVHKHRVVHSHVHKHNVVHSHVHRHQILHRHVHRHQVVHRHVHRHLIAHRHIHSHQAAVHRHVHTHFEGNFNDDGTDVNLRIRHGIIYFGGNTYRLSGGRRRFMTLWQECLESYGDSDECFVQLLEGNQHLFTVVQGHHSTSFRSDLSNDLHPDNNIEQIANDHVNDIAQSTDGDINDFADTHYNDVAPIADVHVDNIAQTADNHVKNIAQTAHHHVNDVAQIADDHVNDIGQTAYDHVNNIGQTADDHVNDIAQTADDHVNAIAQTADDHVNAIAQTADDHVNDIGDTANSHIVRVQGVAKNHLYGINKAIGKHIQHLKDVSNRHIEKLNNHATKNLLQSALQHKQQTIEREIQHKRHLSEKEDINLQHENAMKSKVSYDGPVFNEKVSVVSNQGSYNEKVPVLSNGGGYNGKVSALSDQGSYNEGYAY

SEQ ID NO: 7 - protein, 82 aa, Artificial Sequence
  fp-5
KHHHHHHSSEEYKGGYYPGNTYHYHSGGSYHGSGYHGGYKGKYYGKAKKYYYKYKNSGKYKYLKKARKYHRKGYKKYYGGSS

SEQ ID NO: 8 - protein, 103 aa, Artificial Sequence
  fp-6
IAALCGIVKSIDSDDSDYDYKGRGYCTNKGCRSGYNYFGNKGYCKYGEKSYTYNCNSYAGCCLPRNPYGKLKYYCTNKYGCPNNYYFYNNKGYYYLEHHHHHH

SEQ ID NO: 9 - protein, 199 aa, Artificial Sequence
  fp-151
AKPSYPPTYKAKPSYPPTYKAKPSYPPTYKAKPSYPPTYKAKPSYPPTYKAKPSYPPTYKPWSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYKL

SEQ ID NO: 10 - protein, 171 aa, Artificial Sequence
  fp-131
AKPSYPPTYKAKPSYPPTYKAKPSYPPTYKAKPSYPPTYKAKPSYPPTYKAKPSYPPTYKPWADYYGPKYGPPRRYGGGNYNRYGRRYGGYKGWNNGWKRGRWGRKYYGSAKPSYPPTYKAKPSYPPTYKAKPSYPPTYKAKPSYPPTYKAKPSYPPTYKAKPSYPPTYKL

SEQ ID NO: 11 - protein, 175 aa, Artificial Sequence
  fp-353
PWADYYGPKYGPPRRYGGGNYNRYGRRYGGYKGWNNGWKRGRWGRKYYPWSSEEYKGGYYPGNTYHYHSGGSYHGSGYHGGYKGKYYGKAKKYYYKYKNSGKYKYLKKARKYHRKGYKKYYGGSSGSADYYGPKYGPPRRYGGGNYNRYGRRYGGYKGWNNGWKRGRWGRKYYGS

SEQ ID NO: 12 - protein, 187 aa, Artificial Sequence
  fp-153
AKPSYPPTYKAKPSYPPTYKAKPSYPPTYKAKPSYPPTYKAKPSYPPTYKAKPSYPPTYKPWSSEEYKGGYYPGNTYHYHSGGSYHGSGYHGGYKGKYYGKAKKYYYKYKNSGKYKYLKKARKYHRKGYKKYYGGSSGSADYYGPKYGPPRRYGGGNYNRYGRRYGGYKGWNNGWKRGRWGRKYYGS

SEQ ID NO: 13 - protein, 187 aa, Artificial Sequence
  fp-351
PWADYYGPKYGPPRRYGGGNYNRYGRRYGGYKGWNNGWKRGRWGRKYYPWSSEEYKGGYYPGNTYHYHSGGSYHGSGYHGGYKGKYYGKAKKYYYKYKNSGKYKYLKKARKYHRKGYKKYYGGSSGSAKPSYPPTYKAKPSYPPTYKAKPSYPPTYKAKPSYPPTYKAKPSYPPTYKAKPSYPPTYK

SEQ ID NO: 14 - protein, 60 aa, Artificial Sequence
  fp-1 variant
AKPSYPPTYKAKPSYPPTYKAKPSYPPTYKAKPSYPPTYKAKPSYPPTYKAKPSYPPTYK

SEQ ID NO: 15 - protein, 196 aa, Artificial Sequence
  fp-151 variant
AKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 16 - protein, 76 aa, Artificial Sequence
  mgfp-5
SSEEYKGGYYPGNTYHYHSGGSYHGSGYHGGYKGKYYGKAKKYYYKYKNSGKYKYLKKARKYHRKGYKKYYGGGSS